(19) United States
(12) Patent Application Publication
(10) Pub. No.: US 2020/0013500 A1
(43) Pub. Date: Jan. 9, 2020

(54) CLOUD-BASED SYSTEM FOR CAREGIVER AND CLIENT FOR CREDENTIALING, PATIENT REFERRALS, CLINICAL DOCUMENTATION, AND SCHEDULING

(71) Applicant: Eric Mora, Houston, TX (US)

(72) Inventor: Eric Mora, Houston, TX (US)

(21) Appl. No.: 16/502,632

(22) Filed: Jul. 3, 2019

Related U.S. Application Data
(60) Provisional application No. 62/693,761, filed on Jul. 3, 2018.

(51) Int. Cl. G16H 40/20; H04L 29/06; G06F 21/62; G06F 21/31

(52) U.S. Cl. CPC G16H 40/20; H04L 63/12; G06F 2221/2141; G06F 21/31; G06F 21/6245

(57) ABSTRACT

A caregiver credentialing system may receive, at a server, a request for creation of a caregiver account from a user device. The server may transmit a request for at least one credential for a caregiver associated with the caregiver account to the user device. The server may receive the at least one credential for the caregiver from the first user device and may create a caregiver account for the caregiver based, at least in part, on the receipt of the at least one credential.

### CROSS-REFERENCE TO RELATED PATENT APPLICATIONS

This applications claims the benefit of priority of U.S. Provisional Patent Application No. 62/693,761 filed on Jul. 3, 2018 and entitled “Processes and System that Credentials Caregivers and Clients for Licensed Homecare Within a realtime Map View,” which is hereby incorporated by reference.

### FIELD OF THE DISCLOSURE

The instant disclosure relates to cloud-based credentialing. More specifically, portions of the disclosure relate to cloud-based credentialing of caregivers and clients.

### BACKGROUND

The need for home care in the United States, and worldwide, is projected to increase in coming decades due to a variety of factors, including an aging population. Increased demand for caregivers may outstrip supply in the coming decades, making efficiency in caregiver and client enrollment and assignment an important consideration. Efficiency may be particularly important in rural areas, where caregiver presence is sparse. Lack of flexibility, with rigid scheduling and hours requirements, can make it difficult for caregiver agencies to find caregivers to meet the demand for services. Clients are frequently automatically assigned a caregiver and/or a time slot for caregiver services, unable to select a preferred caregiver at a preferred time.

To provide qualified and competent service, agencies, such as licensed home care agencies, may verify caregiver credentials, making sure that prospective caregivers have appropriate training and a sufficient knowledge base before assigning caregivers to clients. Many agencies also require that clients and caregivers alike agree to one or more agency policies before being enrolled to provide or receive caregiver services. The overwhelming amount of physical paperwork necessary to enroll caregivers and clients can consume valuable time and resources. For example, prospective caregivers may be required to travel to an agency's main office to fill out paperwork for verification of credentials before commencing employment. Furthermore, clients may also be required to fill out and submit paperwork to receive caregiver services, introducing further inefficiency. State and federal laws may require updating of credentials for caregivers and clients at a specified time interval to maintain agency licensure, further contributing to the time consumed filling out and submitting physical paperwork.

Shortcomings mentioned here are only representative and are included simply to highlight that a need exists for improved credentialing systems. Embodiments described herein address certain shortcomings but not necessarily each and every one described here or known in the art. Furthermore, embodiments described herein may present other benefits than, and be used in other applications than, those of the shortcomings described above.

### SUMMARY

A cloud-based caregiver management system may include a cloud-based caregiver and client credentialing system, allowing caregivers and clients to complete enrollment procedures with a user device, such as a laptop, cell phone, tablet, or other personal electronic device. Caregivers and clients may read company policies, enter credential information, and enter personal details with a user device. The information submitted by caregivers and/or clients may be aggregated on one or more remote servers. Once the information is received and, in some cases, verified at a remote server, an account may be created for the caregiver or client. After creation of the account caregiver and client information may be added to a caregiver and client database. Caregivers may be granted access to a caregiver user interface, allowing them to see details of client requests for caregiver services. Clients may be granted access to a client user interface, allowing them to see details of caregivers offering caregiver services. Performing registration and credentialing through a cloud interface may reduce the costs associated with on-site paperwork completion and credentialing and may provide a more positive user experience. Caregivers and clients may be able to register to provide or receive caregiver services from the comfort of their own home, removing the inconvenience and inefficiency of agency headquarters visits and/or delivery of paperwork through the mail.

A caregiver may register with an agency to provide caregiver services to clients of the agency. A method for registering a caregiver may begin with receiving, at a server, a request for creation of a caregiver account from a first user device. For example, a caregiver agency may operate one or more servers for credentialing caregivers and clients and for aggregating caregiver and client data. A caregiver may initiate an application process to create a caregiver account and begin providing services to clients through a portal provided by the agency. For example, a potential caregiver may download and/or open a website, web application, mobile application, personal computer application, or other interface or program for registering as a caregiver.

After receiving the request, the server may transmit, to the user device, a request for at least one credential of the caregiver attempting to create the caregiver account. For example, the agency may require evidence of specific training, certifications, background check information, a government issued identification, education credentials, or other credentials of the caregiver. The agency may require a caregiver to read and agree to one or more agency policies before account creation is finalized. The agency may request an electronic signature from the caregiver, indicating that the caregiver has read and agrees to one or more policies. Thus, a request for one or more credentials may be transmitted from the server, received by the first user device, and displayed to the potential caregiver.

The potential caregiver may enter credentialing information and may submit it to the server. The server may receive the at least one credential for the caregiver from the first user device. The server may receive credentials in a variety of formats, such as text entered by the user, PDFs of credential documents, photographs of credential documents, and other formats. In some embodiments, the server may verify the at least one credential received from the user device. For example, the server may compare the client credential to one or more databases to verify that information submitted by the caregiver is accurate.

After the credentials are received, an account for the caregiver may be created, based on the credential. In some embodiments, the server may require approval from agency personnel before finalizing account creation. When the account is created, the caregiver may be given access to a caregiver user interface for viewing and/or interacting with clients requesting caregiver services. In some embodiments, the caregiver may be given access to a map or list interface, showing clients requesting services. For example, the interface may show clients in proximity to the caregiver, displaying a distance of the client from the current location of the caregiver. In some embodiments, the map interface may also estimate a time it would take for the caregiver to travel from a current location to the location of the client, in order to estimate if the caregiver will be able to arrive during a timeframe requested by the client. The caregiver interface may also allow the caregiver to list their services and expertise for selection by clients looking for caregiver services. For example, the server may add caregiver information associated with the caregiver account to a user interface, such as a map or list interface, accessible by clients of the agency. The caregiver may also receive direct requests from clients interested in the caregiver's services, via the caregiver user interface. Client accounts may be created according to a similar process, and may require credentials such as electronic signatures acknowledging and assenting to one or more caregiver agency policies.

In some embodiments, caregiver credentials may be updated from time to time. For example, an agency may update one or more caregiver policies and may require that a caregiver agree to the updated policies. Federal, state, and/or local government may require that caregiver credentials be renewed periodically, such as yearly. The server may push a notification to a user device associated with a caregiver account, notifying the caregiver that a credential renewal is required and requesting the renewed credential. The caregiver may enter renewed credential information, and the user device may transmit the renewed credential to the server. In some embodiments, the server may block access of the caregiver account to one or more features of the caregiver user interface, preventing the caregiver from providing caregiving services, until credentials are updated. Once the server receives the updated credential, the server may update the caregiver account based, at least in part, on the renewed credential. If access to one or more features of the caregiver interface was blocked, access may be restored once the caregiver account is updated with the updated credentials. Requirements for updated credentials for client accounts may be handled similarly.

Agencies may require caregivers to complete skills training before providing client services or in order to continue providing client services. Skills training may, for example, include educational readings, videos, and/or interactive quizzes. A skills notification may be transmitted by the server to the user device. The caregiver may then complete the skills training. When the skills training is completed, the user device may transmit a confirmation to the server, confirming that the skills training is completed. The skills training completion notification may be received by the server and the server may update the caregiver account accordingly. In some cases, the server may block caregiver access of one or more caregiver user interface features until required skills training is completed. For example, a caregiver may be required to complete specific skills training exercises before providing caregiver services to specific clients, and the caregiver may be blocked from accepting caregiver services requests from such clients until the required skills training exercises are complete.

A client may request caregiver services via a client user interface of a cloud-based caregiver management system. A caregiver services request from a client user device may be received. For example, one or more servers of a cloud-based caregiver management system may receive

A determination may be made based, at least in part, on the caregiver services request of one or more caregiver requirements. For example, a client may request caregiver services that require special training or certifications, such as services that may only be performed by a physical therapist or registered nurse. In some embodiments, requirements for caregivers services may be preloaded on the server in a profile associated with the client.

A determination may then be made of at least one caregiver to match to the client based, at least in part, on the one or more caregiver requirements. The matched caregivers may, for example, be verified to handle caregiver services requests associated with the one or more caregiver requirements. For example, caregivers that are verified to handle caregiver services requests may have special training, certifications, and/or education that correspond to the caregiver requirements. The server that receives the caregiver services request may, for example, have a database of caregivers and associated caregiver credentials, such as trainings, certifications, and/or education, and may match caregivers having credentials that correspond to the caregiver requirements.

Information regarding the at least one matched caregiver may be transmitted to the client user device, for display on the client user device. For example, information for multiple matched caregivers may be transmitted to the client user device, and may be displayed on a map, list, or other interface of the client user device. The client may then select one of the matched caregivers from which to request services. In some embodiments, the caregiver may be assigned to the client upon selection. In some embodiments, a caregiver selection may be received by a server, and a notification of the caregiver services request may be transmitted to a caregiver user device associated with the requested caregiver. An acceptance notification may be received, from the caregiver user device. A confirmation notification may then be transmitted to the client user device that the caregiver has accepted the request for caregiver services.

In some embodiments, the match may include an automatic assignment of a caregiver to the client user device. For example, the server may automatically assign a caregiver with credentials and/or availability that correspond to the caregiver requirements of the caregiver services request to the client. Alternatively the server may transmit caregiver services request information to one or more caregiver user devices for caregivers having verified credentials corresponding to the caregiver requirements of the request. For example, a notification of the request for caregiver services may be transmitted to multiple caregiver user devices and displayed on a map, list, or other interface of the caregiver user devices. The server may receive an acceptance from one or more of the caregivers, may assign the caregiver services request to the caregiver that accepted the request, and may notify the client of the acceptance.

An apparatus for managing a caregiver system may include a memory and a processor configured to perform the steps described herein. A non-transitory computer readable medium may include code for performing the steps described herein. For example, a computer program product may be installed on a server for managing caregiver and client account creation and maintenance.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a diagram of an example system for caregiver and client account registration and maintenance according to some embodiments of the disclosure.

FIG. 2 is an example caregiver user interface with a plurality of potential clients requesting caregiver services according to some embodiments of the disclosure.

FIG. 3 is an example client user interface with a plurality of potential caregivers offering caregiver services according to some embodiments of the disclosure.

FIG. 4A is an example electronic signature credentialing interface according to some embodiments of the disclosure.

FIG. 4B is an example agency policy acknowledgement interface according to some embodiments of the disclosure.

FIG. 5 is an example training interface for viewing of a training video by a caregiver according to some embodiments of the disclosure.

FIG. 6 is a flow chart of an example method for account creation at a server according to some embodiments of the disclosure.

FIG. 7 is a flow chart of an example method for account creation at a user device according to some embodiments of the disclosure.

FIG. 8 is a flow chart of an example method for updating a credential according to some embodiments of the disclosure.

FIG. 9 is an example home page for a client user interface according to some embodiments of the disclosure.

FIG. 10 is an example availability page for a caregiver user interface according to some embodiments of the disclosure.

FIG. 11 is an example navigation menu for a client user interface according to some embodiments of the disclosure.

FIG. 12 is an example agency selection page for a client user interface according to some embodiments of the disclosure.

FIG. 13A is an example care type selection page for a client user interface according to some embodiments of the disclosure.

FIG. 13B is an example extended care type selection page for a client user interface according to some embodiments of the disclosure.

FIG. 14 is an example onboarding page for client account creation according to some embodiments of the disclosure.

FIG. 15 is a flow chart of an example method for processing caregiver services requests at a server according to some embodiments of the disclosure.

FIG. 16 is a flow chart of an example method of generating a caregiver services request at a client user device according to some embodiments of the disclosure.

FIG. 17 is a flow chart of an example method of processing a caregiver services request at a caregiver user device, according to some embodiments of the disclosure.

### DETAILED DESCRIPTION

A cloud-based system for caregiver and client credentialing can allow caregivers and clients to register to provide and receive caregiver services, without the need to on-site paperwork. Caregivers may register to provide caregiver services, submitting required information and credentials, from a user device, such as a personal computer, smart phone, or tablet device. Clients may register to receive caregiver services, providing required information and credentials, from a user device as well. Caregiver and client registration and credentialing may be managed at one or more remote servers to allow caregivers and clients to register from the comfort of their own homes. The cloud-based credentialing system may also allow for upkeep of caregiver and client credentials, pushing notifications of required credential updates to user devices, and, in some embodiments, preventing caregiver and client access of the caregiver services system if credentials are not maintained. The cloud-based credentialing system may be integrated in a larger cloud-based caregiver management system, allowing credentialed caregivers to connect to clients requesting services in their area, and vice versa. The cloud-based caregiver and client credentialing system may reduce the need for costly on-site credentialing visits and may allow caregivers and clients to directly connect to caregivers/clients in their area to provide or receive services.

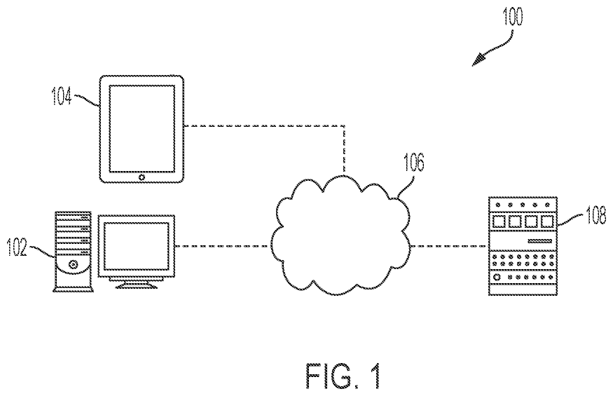

An example cloud-based credentialing system 100 is shown in FIG. 1. A first user device 102 may connect to a cloud-based caregiver services server 108 via a network 106. For example, a caregiver services web application may be executed either on the server 108, on the user device 102, or with operation distributed across both the server 108 and the user device 102. Alternatively or additionally, a local caregiver services application may operate on user device 102, receiving information input by a user and exchanging information with the server 108. Additional user devices, such as second user device 104 may also connect to the server 108 via a network 106. Although only a single server 108 is shown, multiple user devices may connect to multiple servers through the network 108. In some cases, operation of a caregiver services web, or other, application may be distributed across multiple servers.

User device 102 may, for example, be a user device operated by a caregiver. A caregiver may operate the user device 102 to connect to the server 108 and create an account to provide caregiver services. The server 108 may request information from the caregiver user device 102 required for creation of an account. For example, the server may request personal information, such as name, location, hours of availability, and other personal information, and credentials. Credentials may, for example, include certifications, government-issued identification, transcripts, evidence of one or more degrees, background check information, verification that the caregiver has read and assents to one or more policies of a caregiver agency operating the system, and other credentials. A caregiver requesting account creation may enter personal information and credentials via the user device 102, and the user device 102 may transmit the personal information and credentials to the server 108. For example, the caregiver may take pictures of a government issued identification, a diploma, a certification, or other credential with the user device 102 and may submit them to the server 108. Credentials may also be submitted in the form of scanned documents, PDFs, electronic signatures, and other formats. After the caregiver submits personal information and/or credentials, the server 108 may create a caregiver account for the caregiver. The caregiver user device 102 may also be operated to accept requests for caregiver services, and the server 108 may transmit caregiver services requests to and receive caregiver services requests from the caregiver user device 102.

User device 104 may, for example, be a user device operated by a client. A client may operate the user device 104 to connect to the server 108 and create an account to obtain caregiver services. The server 108 may request information from the client user device 104 required for creation of the account. For example the server may request personal information, such as name, location, type of requested care, hours for which care is requested, and other personal information, and credentials, such as verification that the client has read and assents to one or more policies of a caregiver agency operating the system and insurance and payment information. For example, the client may read policies issued by the caregiver agency and may submit an electronic signature acknowledging and assenting to the policies. After the client submits personal information and/or credentials, the server 108 may create a client account for the client. The client user device 104 may also be operated to request caregiver services, and the server 108 may receive caregiver services requests from the client user device 104.

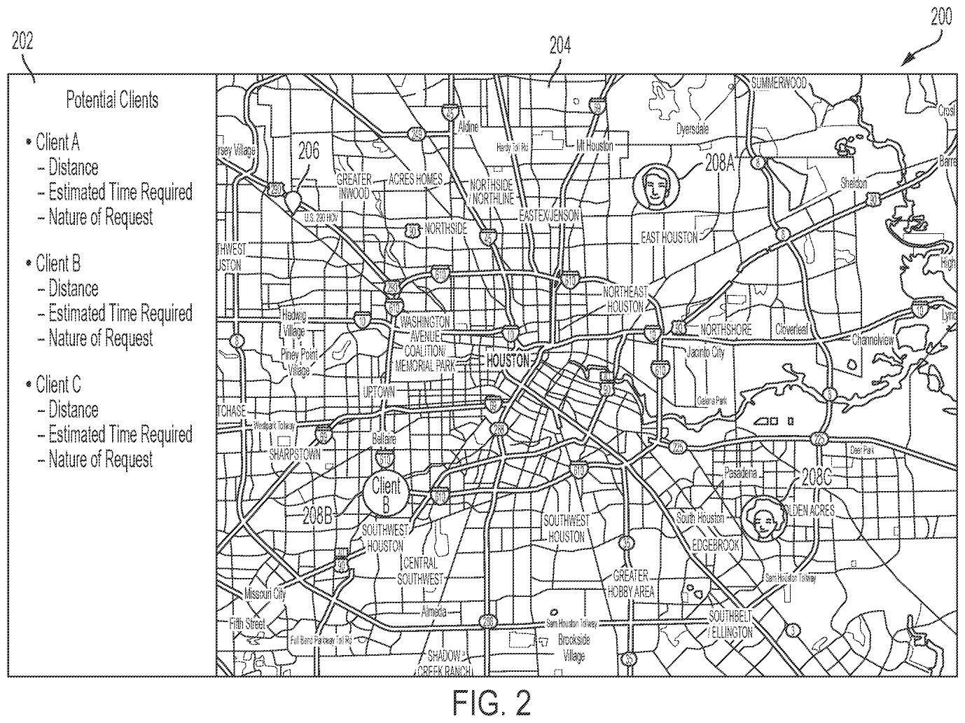

After a caregiver or client account is created, the account may be granted access to a caregiver or client user interface. The user interface may, for example, be generated and displayed via a cloud-based web application accessible by clients and caregivers, via a local application on a user device, or via another application. An example caregiver user interface 200 is shown in FIG. 2. Caregivers may be restricted from accessing the caregiver user interface until caregiver registration is complete, and the caregiver's credentials have been verified. The caregiver user interface 200 may, for example, include a map interface 204, showing a plurality of potential clients 208A-C within a specified region, requesting caregiver services. For example, the map interface 204 may show a plurality of potential clients within a specified distance of a location of the caregiver 206 requesting caregiver services. The map interface 204 may be a real time map view, displaying current locations of a plurality of clients. In some embodiments, clients may be filtered based on a time required for the caregiver to arrive at the client. For example, clients may request services within a specified time frame. A remote server generating the user interface may calculate a time it would take for the caregiver to reach the client based on map data, traffic data, and/or a distance of the caregiver from the client, and may display clients which the caregiver may reach before a requested time window for caregiver services expires. Clients displayed on the map interface 204 may be filtered according to a specialty of the caregiver such that only clients requesting specialty services from a caregiver that correspond to a specialty of the caregiver may appear. For example, clients may have needs requiring specialized care, and such clients may be displayed only for caregivers with certifications in providing such specialized care and/or who have completed specified training in providing the specialized care. Clients displayed on the map interface 204 may also be filtered based on caregiver preferences, such as a match between caregiver and client availability windows. The map interface 204 may also show a picture of a potential client, such as shown for clients 208A, 208C, or a name of a client, such as shown for client 208B. Alternatively or additionally, a list interface 202 of clients requesting caregiver services may be shown. The list interface 202 may also show information such as distance, an estimated time of arrival, and a nature of the caregiving services requested by the client. The list interface may be filtered similarly to the map interface. In some embodiments, the caregiver user interface 200 may display a list of clients that have specifically requested services from the caregiver. Caregivers may select clients from either the list interface 202 or the map interface 204 to see additional details of a request and to offer their services. For example, caregivers may view a list of available shift times and dates in either a list or map view and accept shifts that work with their schedule. In some embodiments, the client request may be automatically assigned to the caregiver upon acceptance, while in other embodiments, information about the caregiver may be sent to the client for approval, prior to assigning the client request to the caregiver. The caregiver user interface 200 may also include a calendar, showing booked client appointments and available time to schedule additional client appointments. A server of the caregiver system may also issue push notifications to a caregiver's user device, presented via a caregiver user interface, when a client has requested the caregiver's services.

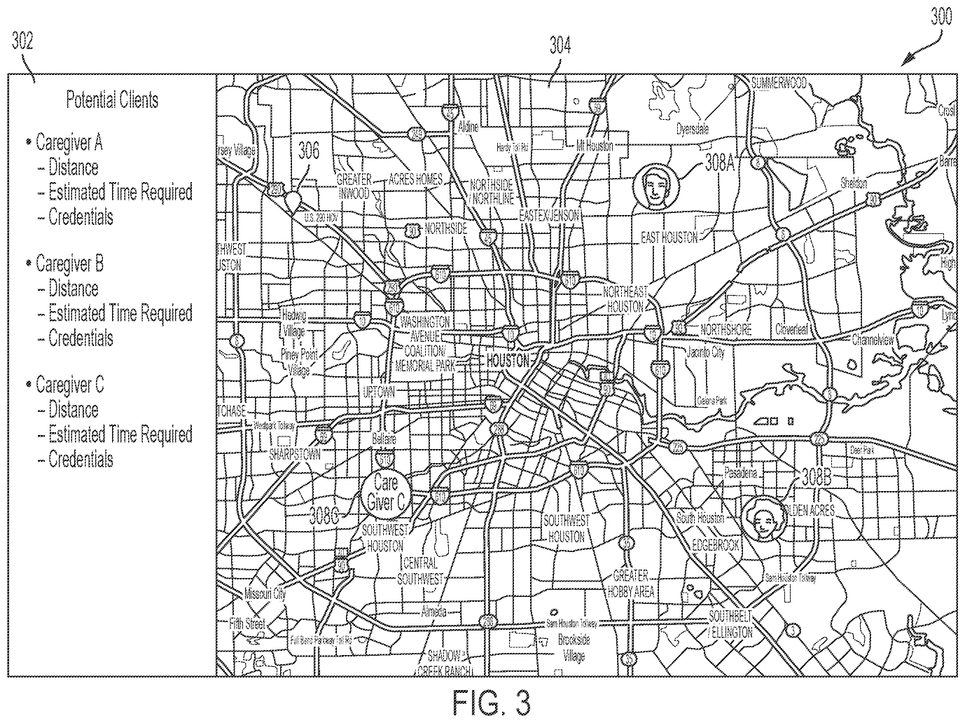

An example client user interface 300 is shown in FIG. 3. Clients may be restricted from accessing the client user interface until they have agreed to one or more caregiver agency policies and provided relevant client information, such as payment and/or proof of insurance. The client user interface 300 may, for example, include a map interface 304, showing a plurality of caregivers 308A-C within a specified region, available to provide caregiver services. For example, the map interface 304 may show a plurality of potential caregivers within a specified distance of a location of the client 306 that are available to provide caregiver services. The map interface 304 may be a real time map view, showing a real time location of one or more caregivers. In some embodiments, caregivers may be filtered based on a time required for the caregiver to arrive at the client. For example, the client may request services within a specified time frame. A remote server generating the user interface may calculate a time it would take for caregivers within a specified range to reach the client based on map data, traffic data, and/or a distance of the caregiver from the client, and may only display caregivers that are able to reach the client before a requested time window for caregiver services expires. Caregivers displayed on the map interface 304 may be filtered according to specialty. For example, the client may request services requiring a caregiver with specialized training or certifications, and caregivers shown may be limited to caregivers having the required training and/or certifications. The map interface may also show a picture of a potential caregiver, such as shown for caregivers 308A, 308B, or a name of a caregiver, such as shown for caregiver 308C. Alternatively or additionally, a list interface 302 of caregivers available to provide caregiver services may be shown. The list interface 302 may also show information such as distance, an estimated time of arrival, and any specialized training, certifications, or skills the caregiver may have. The list interface 302 may be filtered similarly to the map interface. In some embodiments, the client user interface 300 may display a list of caregivers that have specifically offered their services to the client. When a client selects a caregiver, the user interface may display a caregiver calendar, showing the caregiver's working hours and/or availability of the caregiver to provide client services. If a client wishes to request services from a specific caregiver, the client may book a time slot. In some embodiments, the caregiver may be automatically assigned to the client at that time slot following the client's request, while in other embodiments caregiver approval may be required to finalize an appointment. A server of the caregiver system may also issue push notifications to a caregiver's user device, presented via a caregiver user interface, when a client has requested the caregiver's services. Thus a map and/or list user interface may allow clients and caregivers to easily connect for provision of caregiver services. In some embodiments, the caregiver and client user interfaces may hide specific client and/or caregiver information until a caregiver and client have agreed to work together to maintain privacy. In some embodiments, the interface may allow messaging between a caregiver and a client, such as via a web-application.

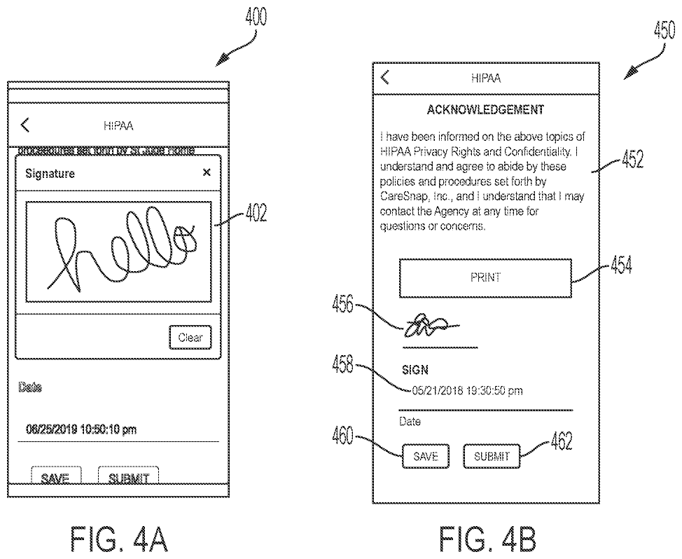

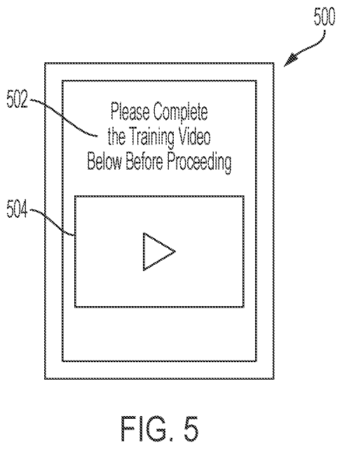

Various credentials may be required from caregivers and clients to create and maintain an account. For example, caregivers may be required to submit licenses, certifications, state issued identification, proof of education, background check information, and electronic signatures assenting to various caregiver agency policies. Clients may also be required to submit credentials such as proof of insurance, payment information, and electronic signatures assenting to various caregiver agency policies. The credentialing system may also require that credentials be updated periodically. For example, the system may require that clients and/or caregivers review company policies and submit electronic signatures verifying that the caregiver or client has read and assents to the policies at a specified interval, such as every year, or when material changes are made to one or more policies. A server of a caregiver credentialing system may transmit a push notification to a user device associated with a caregiver or client account notifying the caregiver or client that an updated credential is required. The device may display one or more policies that the user or caregiver is required to read. After the polices are read, an electronic signature may be required. A user device 400, shown in FIG. 4A, may display an electronic signature page. The electronic signature page may include a region 404 for inputting an electronic signature. The region 404 may, for example, be an interface for inputting an electronic signature via a touch interface, via typing, or via another input method. An electronic signature may, for example, be required from a caregiver or client acknowledging an agency's Health Insurance Portability and Accountability Act (HIPAA) policy. FIG. 4B shows an example user device display of an agency policy acknowledgement page 450. The acknowledgement page 450 may, for example, include text indicating that a caregiver/client has read and agrees to an agency policy. The acknowledgement page 450 may further include a button 454 for printing the one or more policies and/or an electronic signature entered as described with respect to FIG. 4A. The acknowledgement page may include an option to enter an electronic signature, acknowledging and assenting to the agency policy, as described with respect to FIG. 4A, and a copy 458 of the electronic signature once the electronic signature has been entered via the electronic signature page. The acknowledgement page 450 may include a date and time at which an electronic signature was entered. The acknowledgement page of the caregiver management system may include a save button 460 to save an entered electronic signature and other entered information and a submit button 462 top submit the electronic signature and, in some cases, other entered information. When a user submits an electronic signature, the user device may transmit the electronic signature to a server operating the credentialing system. The server may store the electronic signature, along with the policies that the caregiver or client has agreed to, and may update the client or caregiver account with which the electronic signature is updated. For example, credentialing documents may be securely stored across one or more servers in the cloud. In some embodiments, the system may block access by a user device to one or more features of the cloud-based caregiver management system, and, in some cases, to a client or caregiver user interface entirely, until updated credentials are received. Thus, clients and caregivers that have not updated credentials as required may be prevented from booking caregiver services.

In some embodiments, the cloud-based caregiver services system may require caregivers to view training materials, in order to improve caregiver services. For example, caregivers may be required to view video and written training materials and/or complete training quizzes periodically in order to continue providing caregiver services, or initially, in order to begin providing caregiver services to clients requiring specialized services. A remote server of the caregiver services system may push a skills training request to a caregiver, notifying the caregiver that a required training is available. In some embodiments, training may be tailored to a client's plan of care, and the caregiver must complete training prior to providing care to the client. A user device 500 may display a training video 504 with instructions 502. In some embodiments, the caregiver services system may block the caregiver account from accessing one or more features of the cloud-based caregiver management system, such as a caregiver user interface for accepting client requests, until the training video has been viewed. In some embodiments, caregivers may be blocked from offering services to clients requiring specialized services until specialized training is complete. When the training video has been viewed, the user device 500 may transmit a notification to a remote server, and the remote server may update a caregiver account of the caregiver. Updating the caregiver account may include restoring access to a caregiver user interface, such as the interface described with respect to FIG. 2.

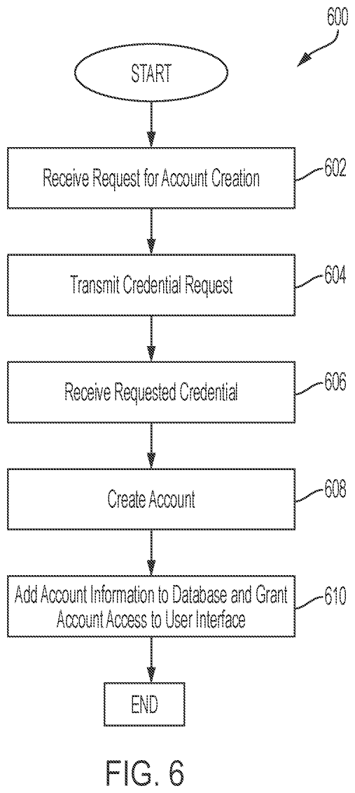

A cloud-based system for caregiver and client credentialing may allow caregivers and clients to register to provide/receive caregiver services without requiring an on-site visit to a caregiver agency location or completion and transportation of physical paperwork. A method 600 for account creation in a cloud-based caregiver and client credentialing system is shown in FIG. 6. The method 600 may begin, at step 602 with receiving a request for account creation. A server may, for example, receive a request for account creation from a user device operated by a caregiver or user. In some embodiments, the request may include caregiver or client information, such as a name, address, date of birth, types of services requested/provided, and other information. For example, the account creation request may include responses to a caregiver or client questionnaire including personal information. With respect to creation of caregiver accounts, the request for account creation may include application materials filled out by the caregiver, such as an application for employment. In some embodiments, the request may also include caregiver bank account information, so that paychecks from a caregiver agency operating the system may be direct deposited.

At step 604, the server may transmit a credential request. For example, a server may transmit a credential request to the user device from which the request for account creation was received requesting one or more caregiver or client credentials. In the case of caregiver account creation, the server may transmit a request for licenses, certifications, state issued identification, proof of education, background check information, and electronic signatures assenting to caregiver agency policies, among other credentials. The server may also request that the caregiver read or view training materials and/or complete one or more quizzes or other skills training tasks and transmit verification of completion. In the case of client account creation, the server may transmit one or more company policies and a request for an electronic signature verifying that the client has read and assents to the one or more policies. In some embodiments, the server may transmit a request for other client credentials, such as proof of insurance and/or payment information.

The caregiver or client may input credentials on the user device and submit the credentials to the server. For example, credential information may be entered on the user device via a keyboard or touch interface. Alternatively or additionally, an image of credential information may be captured via scanning or a camera of the mobile device and submitted. In some embodiments, documents and PDFs, such as academic transcripts and/or copies of certifications may also be submitted. At step 606, the server may receive the requested credentials from the user device. In some embodiments, the server may verify requested credentials, such as by comparing information in the requested credentials to one or more databases. A criminal background check of caregivers requesting caregiver account creation may also be performed.

At step 608, an account may be created. For example, after requested credentials are received and, in some cases, verified, an account may be created for the caregiver or client. At step 610, after an account is created, information may be added to an account database, and the account may be granted access to a user interface. For example, caregiver information may be added to a caregiver account database for presentation to potential clients. Clients may view the caregiver account information on a client user interface, such as a list or map interface, and may select caregivers from which to request services. Client information may be added to a client account database, for presentation to potential caregivers. Caregivers may view client information and may accept client caregiver services requests. After account creation, the cloud-based caregiver system may allow for payment to be seamlessly transferred from clients, or insurer of clients, to caregivers, often within a matter of days of the caregiver providing caregiver services. For example, caregivers may have the option of receiving automated clearing house (ACH) payments within the web, or other, application. For example, when a caregiver completes a caregiver services engagement, the caregiver may fill out and submit a clinical documentation within the caregiver user interface, such as via the caregiver management system web, or other, application. The clinical documentation may be submitted to a the agency with which the caregiver is associated/employed. Once the clinical documentation is approved by the agency, the caregiver may be paid through the application. In some embodiments, the caregiver may be paid on a daily basis.

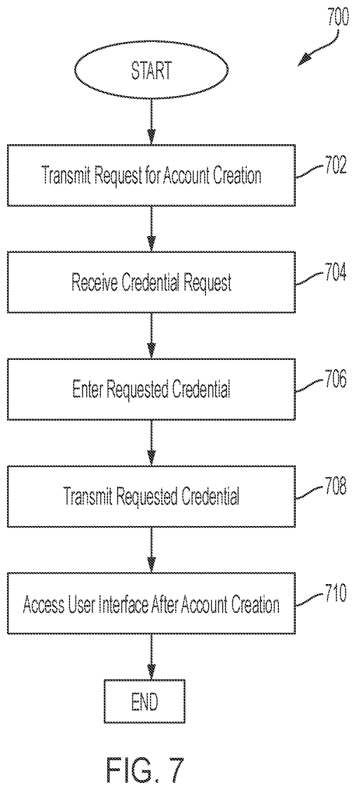

A client or caregiver may register for an account via a user device. An example method 700 for registering with a user device is shown in FIG. 7. The method 700 may begin, at step 702, with transmitting a request for account creation. The user device may, for example, be a smart phone, desktop, tablet, laptop, or other user device. In some embodiments a caregiver or client may download a caregiver services application to initiate account creation. In other embodiments, the caregiver or client may access a cloud-based caregiver services web application for account creation. A caregiver or client may request to create an account, via a user device, and the request may be transmitted to a remote server. In some embodiments, the request may include caregiver or client information, such as a name, address, date of birth, types of services requested/provided, and other information. In some embodiments, caregivers may enter bank account information, so that paychecks from a caregiver agency running the system may be direct deposited.

At step 704 a credential request may be received at the user device. For example, after a request for account creation is received by the server, the server may request one or more credentials from the client or caregiver. In the case of caregiver account creation, the request may include a request for licenses, certifications, state issued identification, proof of education, and electronic signatures assenting to various caregiver agency policies, among other credentials. In the case of client account creation, the request may include one or more company policies and a request for an electronic signature verifying that the client has read and assents to the one or more policies. In some embodiments, other client credentials may be requested, such as proof of insurance and/or payment information, such as insurance automated billing information.

At step 706, requested credentials may be entered on the user device. For example, credential information may be entered on the user device via a keyboard or touch interface. Alternatively or additionally, an image of credential information, such as an image of a government issued identification, may be captured via scanning or a camera of the mobile device and submitted. In some embodiments, documents and PDFs, such as academic transcripts and/or copies of certifications, may also be submitted.

At step 708, the requested credentials that have been entered on the user device may be transmitted to the server. The server may then create a user account following receipt of all requested credentials. In some embodiments, the server may verify requested credentials and may request additional credentials if received credentials do not meet one or more credentialing requirements. For example, if an image of a scanned government issued identification is of insufficient quality, the server may transmit a request to a user device requesting that the user re-enter and transmit the government issued identification. At step 710, after the account is created, the caregiver or client may access a user interface. For example, a caregiver may access a caregiver interface, such as map or list interface as described with respect to FIG. 2, and accept client requests of caregiver services. A client may access a client interface, such as a map or list interface as described with respect to FIG. 3, and request caregiver services. After the account is created, the user device may begin uploading real time location data for the caregiver or client. The real time location data for a caregiver account may be displayed on a client user interface, and the real time location data for a client account may be displayed on a client user interface. In some embodiments, clients and caregivers may configure whether they wish to share real time location information with the caregiver services system and/or with other caregivers and clients. A client may be prompted to schedule an initial in-home assessment following creation of a new account. Clients may be given access to a medical purchases interface for directly ordering durable medical equipment and/or dropship prescription medications from within the web, or other application. In some embodiments, clients may be able to book transportation to and from medical appointments within the web, or other, application. For example, clients may access common e-commerce or ride-sharing services from within the web, or other, application. The system may also grant doctors and/or clients to upload clinical notes and doctor's orders to the client account, so that caregivers can provide better services to clients.

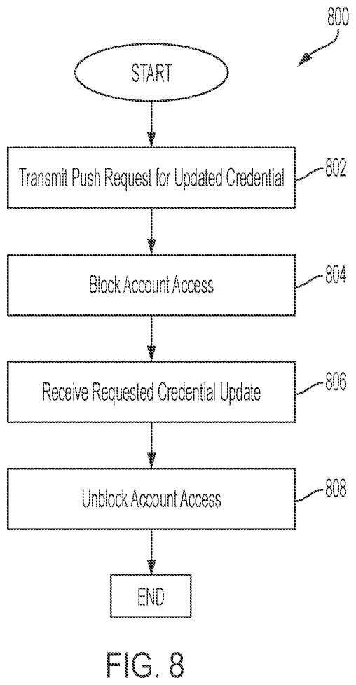

Caregiver and client credentials may be updated periodically, in accordance with caregiver agency policies or government rules and regulations. Caregivers and clients may be required to update assent to caregiver agency policies at a specified interval, such as yearly, or when material changes are made to the policies. For example, caregivers and clients may be required to review and consent to caregiver agency policies on an annual basis. Alternatively or additionally, caregivers may be required to complete caregiver trainings periodically. For example, caregivers may be required to view training videos, read training updates, and/or complete training quizzes, among other training formats before providing specialized services to clients or in order to continue providing services to clients. Caregivers and clients may also be required to update other credentials, such as insurance information and government issued identifications. A method 800 for updating a credential of a client or caregiver is shown in FIG. 8. The method 800 may begin, at step 802, with transmitting a push request for an updated credential. The push request may be transmitted from a server to a user device. For example, a server may determine that a year has passed following a prior policy credential update and may transmit a push request to a user device associated with the client or caregiver account requiring the update instructing the client or caregiver to review one or more caregiver agency policies and submit a renewed electronic signature, indicating that the client or caregiver has read and assents to the one or more policies.

At step 804, the server may block access of the account to one or more features of the cloud-based caregiver management system. For example, when a caregiver account requires updated caregiver credentials, the server may block access of the account to an interface for accepting caregiver services requests. When a client account requires updated caregiver credentials, the server may block the account from requesting caregiver services. In some embodiments, the server may allow continued access of all features of the caregiver system, even when a credential update is required.

After the request for updated credentials is received at the user device, the user may enter updated credentials, such as an electronic signature verifying that the user has reviewed and assents to one or more agency policies. At step 806, the server may receive the requested credential update. At step 808, after the requested credential update is received, the server may restore account access to all aspects of the caregiver management system. Thus, after renewing credentials, caregivers may resume accepting client requests for caregiver services, and clients may resume requesting caregiver services. Such blocking may help to enforce caregiver credentialing compliance.

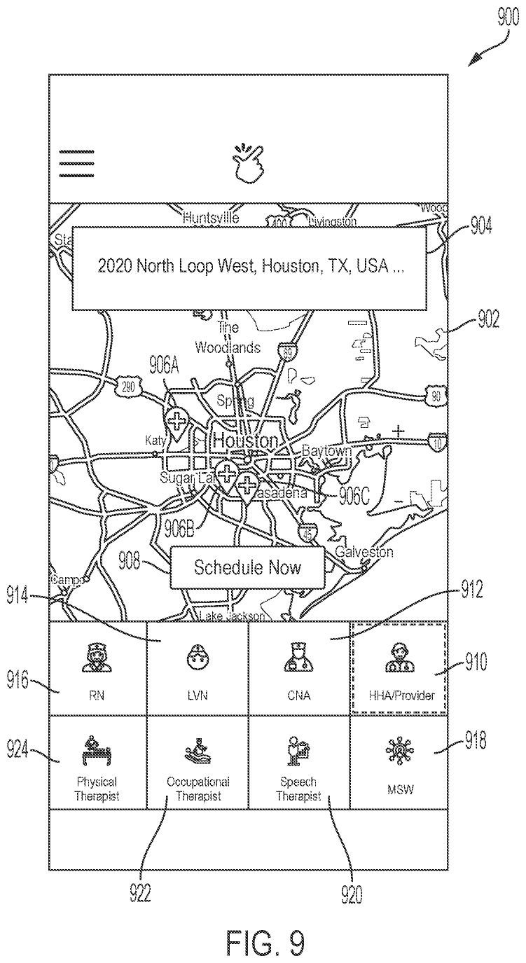

Clients may be able to request different kinds of care from caregivers with different credentials. An example client user interface 900 for a cloud-based caregiver management system is shown in FIG. 9. The client user interface 900 may allow a user to enter a location, via a keyboard, at location search block 904, or may use an internal GPS or other location mechanism of a user device of the client to determine client location. A client may select a type of caregiver from which the client would like to receive services. For example, a user may select a type of caregiver from multiple caregiver types with different types of credentials, such as a home health aide/provider option 910, a certified nursing assistant option 912, a licensed vocational nurse option 914, a registered nurse option 916, a social worker option 918, a speech therapist option 920, an occupational therapist option 922, and a physical therapist option 924. When a client selects one of options 910-924, multiple available caregivers with the requested credentials may appear on the map interface 902. For example, if a client selects the home health aide/provider option 910 multiple available home health aides within proximity of the client may be displayed on the map. For example, home health aides 906A, 906B, and 906C may be displayed on the map. The client may then choose to schedule one of the available home health aides by selecting a scheduling option 908. In some embodiments, the client may select one of the home health aides shown on the map and schedule a specific home health aide. When a client requests caregiver services, the caregiver management system may notify one or more caregivers of the request via text, push notification, email, or another notification method. For example, if a client requests caregiver services from a specific caregiver, the system may send a text message to the caregiver notifying the caregiver that their services have been requested. Alternatively, if a client makes a general request for caregiver services, the system may send a text message to multiple caregivers, notifying the caregivers that a client has requested caregiver services. In some embodiments, a text message may be sent to caregivers in close proximity to the client that have specialized training and/or certifications required by the nature of the client's caregiver services request.

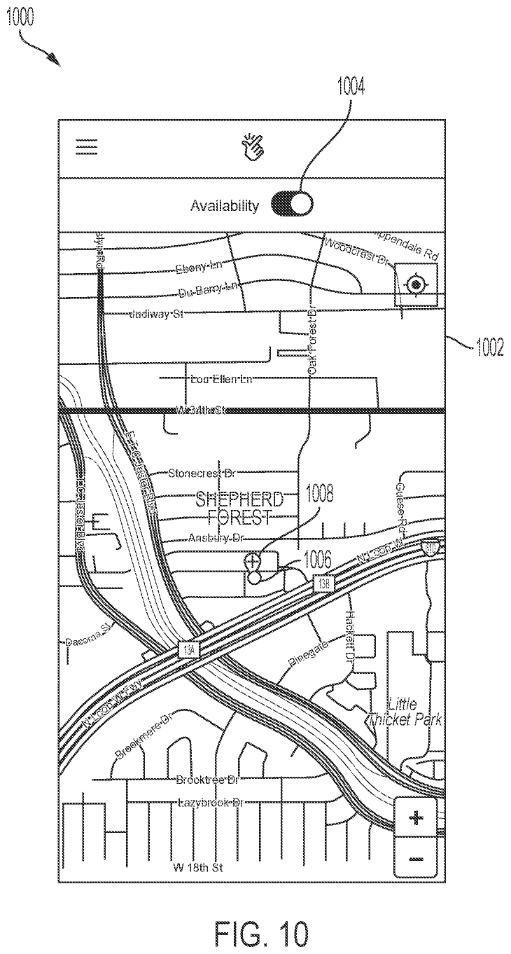

In order to provide flexibility, caregivers may toggle availability on and off at their own convenience. An example caregiver availability configuration interface 1000 is shown in FIG. 10. The caregiver availability configuration interface 1000 may include a map 1002 showing a caregiver location 1006, along with an indicator 1008 as it would appear on a client user interface. The caregiver availability configuration interface 1000 may include an availability toggle 1004. When the caregiver is available to provide clients with caregiver services, the caregiver may switch the toggle 1004 to on. The caregiver may then be listed when clients search for caregivers that are currently available to provide caregiver services. When the caregiver is not available to provide clients with caregiver services, the caregiver may switch the toggle 1004 to off and the caregiver may be removed from display to clients searching for caregivers currently available to provide caregiver services. Thus, a caregiver may make their information available to clients when the caregiver is available, and the caregiver may hide their information when the caregiver is unavailable.

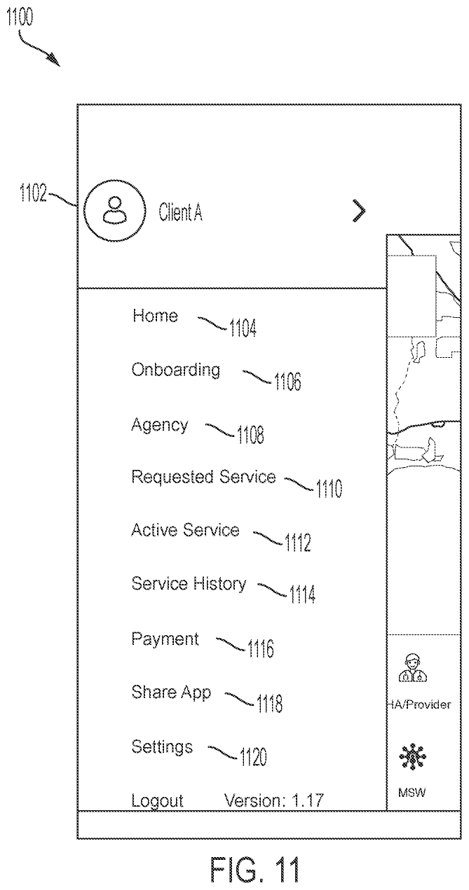

An example client navigation menu 1100 for a client user interface of a caregiver management system is shown in FIG. 11. The client navigation interface 1100 may include a client descriptor 1102 that may include a name and/or photograph of the client. The navigation interface 1100 may also include a home option 1104, for navigating to a home page to request caregiver services, an onboarding option 1106, for registering to receive caregiver services and/or for renewing client credentials, an agency option 1108, for selecting an agency from which to receive caregiver services, and a requested service option 1110, to view a list of caregiver services requested. The navigation interface 1100 may also include an active service option 1112, to view caregiver services requested by the client that are currently active, a service history option 1114, to view past caregiver services received by the client, a payment option 1116, to enter and adjust payment information and to make payments for caregiver services, a share application option 1118, to recommend the application to other potential clients, and a settings option 1120 to adjust settings of the caregiver services management system application.

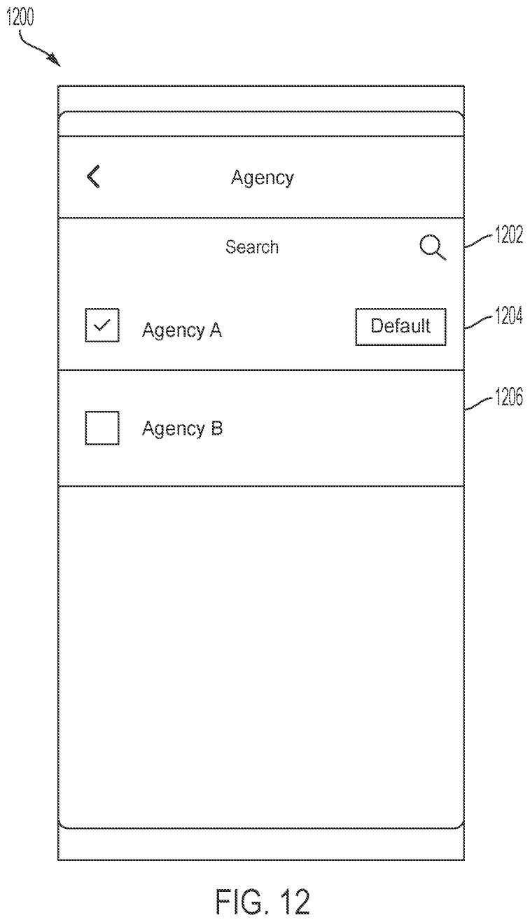

In some embodiments, the caregiver services management system may be operated by a third party, and a client may have an option to select an agency from which to receive caregivers services within a client user interface. An example client user interface 1200 for selecting an agency is shown in FIG. 12. A client may search for client agencies by name using the search box 1202. Alternatively or additionally, agencies with close proximity to the client may appear in list form and the client may select a nearby agency. A client may select from a plurality of agencies, such as a first agency option 1204 and a second agency option 1206. A client may select one of the agency options, such as agency option 1204, as a default agency option. When the client selects an agency from which to receive caregiver services, the caregiver services management system may limit display of available caregivers on the client user interface to caregivers associated with and/or employed by the selected agency. The caregiver services management system may also send referrals to state licensed home care or home health agencies. For example, if a client registers without a preferred agency, the system may refer the client to one or more agencies. In some embodiments, the system may refer a client to one or more agencies when the client requests caregiver services. For example, when a client registers or requests caregiver services without a preferred agency, the caregiver management system may send a text message, push notification, email, or other notification for a client referral to one or more home care or home health services agencies.

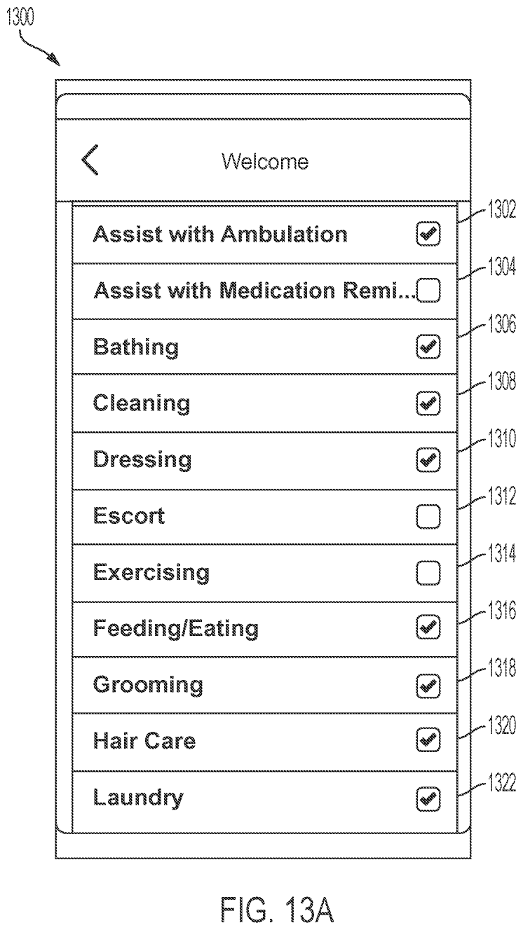

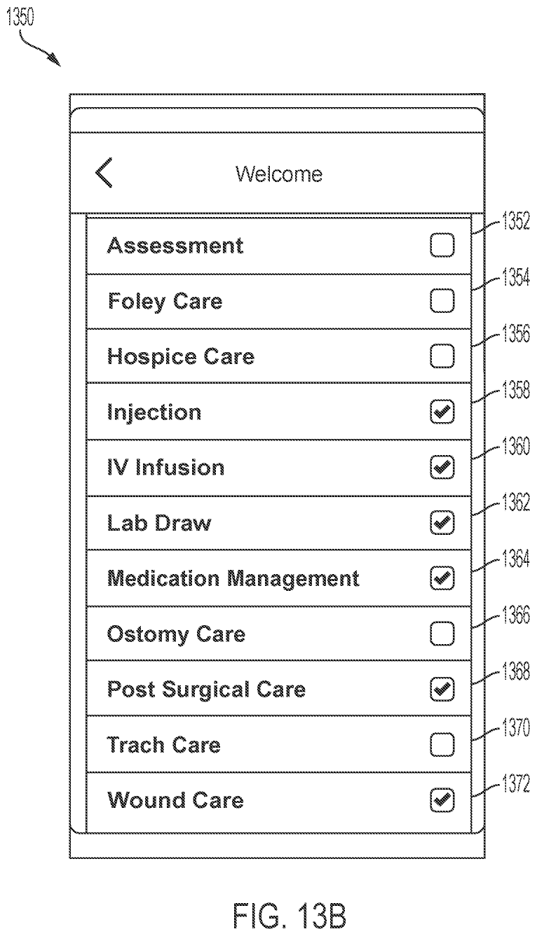

A client may also select a type of caregiver services needed in order to be connected with caregivers having specialized training and/or certifications. For example, a client may enter caregiver services types when registering for a client account and/or when requesting caregiver services. An example client care type selection page 1300 is shown in FIG. 13A. A client may select from a variety of caregiver services options including an ambulation asset option 1302, a medication reminder assist option 1304, a bathing option 1306, a cleaning option 1308, a dressing option 1310, an escort option 1312, an exercising option 1314, a feeding and/or eating option 1316, a grooming option 1318, a hair care option 1320, and a laundry option 1322. Additional options may also be displayed for selection by a client, as shown in extended care type selection page 1350 of FIG. 13B. For example, a client may also select from among an assessment option 1352, a foley care option 1354, a hospice care option 1356, an injection option 1358, an intravenous infusion option 1360, a lab draw option 1362, a medication management option 1364, an ostomy care option 1366, a post-surgical care option 1368, a trach care option 1370, and a wound care option 1372. Other options may also be displayed in addition to those listed. When a client selects care options, caregivers qualified to provide the kinds of caregiver services specified, such as caregivers with specialized training and/or certifications, may be recommended to the client. For example, when a client requests caregiver services via a map, list, or other interface, the map interface may limit display of available caregivers to available caregivers that have specialized training or certifications related to the specific requested caregiver services.

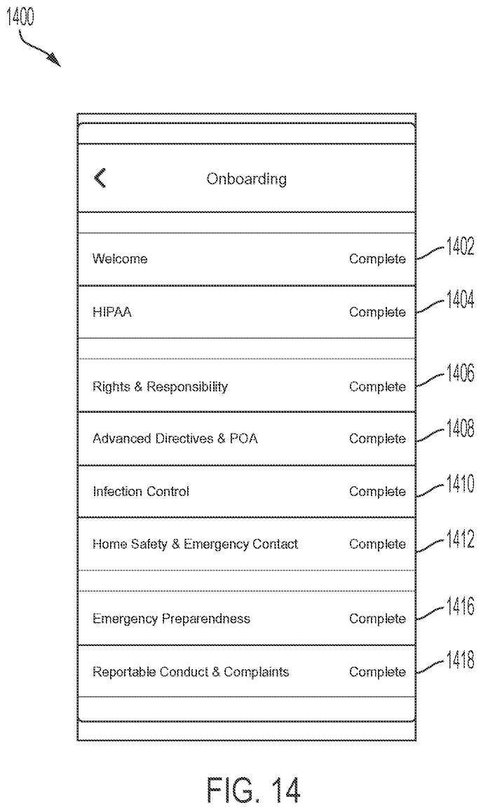

Clients and caregivers may be required to agree to a variety of agency policies in order to register to receive/provide caregiver services. An example policy onboarding page 1400 for a potential client is shown in FIG. 14. A client may be required to read and provide electronic signatures for a variety of agency policies. For example, a client may be required to read a welcome page 1402. A client may be required to read and provide an electronic signature for a HIPAA policy 1404, a rights and responsibility policy 1406, an advanced directives and power of attorney policy 1408, an infection control policy 1410, a home safety and emergency contact policy 1412, an emergency preparedness policy 1416, and a reportable conduct and complaints policy 1418. A potential client may also be required to enter additional information when reading and providing electronic signatures for policies, such as emergency contact information and power of attorney information. Caregivers may also be required to read and provide electronic signatures assenting to a similar array of agency policies.

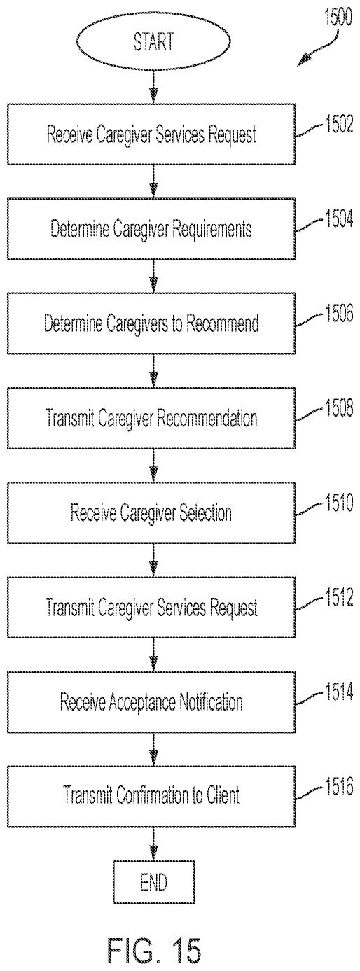

A server in a cloud-based caregiver management system may process requests for caregiver services. An example method 1500 of processing a request for caregiver services is shown in FIG. 15. The method 1500 may begin, at step 1502, with receipt of a caregiver services request. For example, a server may receive a caregiver services request from a client user device. The caregiver services request may, in some embodiments, include information regarding a nature of the caregiver services requested and a time frame during which caregiver services are requested. In some embodiments, the caregiver services request may be a request for services from a specific pre-approved caregiver, and the server may automatically assign the request to the requested caregiver.

At step 1504, the server may determine caregiver requirements associated with the caregiver request. For example, the caregiver services request may specify specific services requested, such as those described with respect to FIGS. 13A-B. Alternatively, specific services requested may be associated with the client account and may be accessed from a client account database. Thus, the caregiver services request may require that a caregiver be capable of performing and/or have training, education, and/or certifications associated with the requested services. Alternatively or additionally, a caregiver services request may include a request for a caregiver with specific credentials, such as a registered nurse, a physical therapist, a certified nursing assistant, or other a caregiver with other credentials.

At step 1506, the server may determine one or more caregivers to recommend. For example the server may match caregivers with certain characteristics to the client. For example, the server may search a database of caregivers to locate caregivers that are active during the requested time period. The server may further filter potential caregivers by locating caregivers that are verified for the one or more caregiver requirements of the caregiver services request. A caregiver may be verified for the one or more caregiver requirements if the caregiver has one or more credentials, such as specialized training, certifications, and/or education, that fulfill the caregiver requirements associated with the caregiver services request. For example, caregiver credentials may be submitted by the caregiver during or following the caregiver account creation process. In some embodiments, the server may transmit the caregiver services request to multiple caregivers having credentials associated with the one or more caregiver requirements. The request may be displayed on a map, list, or other interface of the caregiver user devices of the selected caregivers. One or more of the selected caregivers may accept the request, and a notification of acceptance of the request may be transmitted to the server. The server may then assign a caregiver that accepted the request to the client, and may notify the client that they have been assigned a caregiver for the request.

At step 1508, the server may transmit the recommendation of one or more caregivers to the client user device. In some embodiments, the recommended caregivers may be displayed on a map, list, or other interface for viewing by the client. The client mays select a caregiver from among the recommended caregivers, and the client user device may transmit a caregiver selection to the server.

At step 1510, the server may receive a caregiver selection from the client user device. In some embodiments, the server may automatically assign the caregiver to the caregiver services request upon receipt of the caregiver selection from the client user device, while in other embodiments, the server may seek approval from the selected caregiver.

At step 1512, the server may transmit a caregiver services request to a caregiver user device associated with the selected caregiver. The caregiver may accept the request and may transmit an acceptance notification to the client user device. At step 1514, the server may receive the acceptance notification from the client user device, and at step 1516, the server may transmit a confirmation that the caregiver has accepted the clients caregiver services request to the client user device. Thus, a server may handle requests for caregiver services from a client and may match clients with caregivers verified to handle the specific requests of the client.

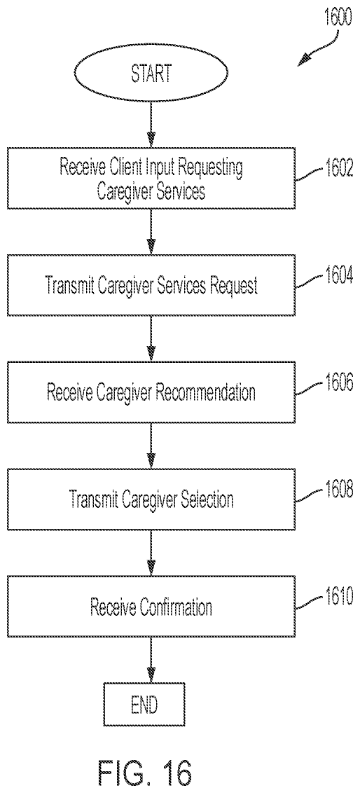

A client may request caregiver services via a client user device and may be matched with caregivers verified to handle the specific requests of the client. A method 1600 for requesting caregiver services with a client user device is shown in FIG. 16. The method 1600 may begin, at step 1602, with receipt of client input requesting caregiver services. For example, a client may specify a nature of requested caregiver services, as described with respect to FIGS. 13A-B, desired training and or certifications of any caregivers to be recommended, a desired time frame during which the client wishes to receive caregiver services, and other caregiver services request details. At step 1604, the caregiver services request may be transmitted from the client device to a server for handling caregiver services requests. In some embodiments, the server may respond by automatically assigning a caregiver to fulfill the clients caregiver services requests, while in other embodiments, as described with respect to steps 1616-1610, the server may provide multiple recommendations of caregivers to provide the caregiver services.

At step 1606, the client user device may receive a recommendation of one or more caregivers to provide the requested caregiver services. The recommendation may include information such as a caregiver location, name, picture, certifications, and other caregiver information. In some embodiments, the received caregiver information may be displayed on a map, list, or other interface, for selection by the client. The client may select one of the recommended caregivers from which to receive caregiver services, and, at step 1608, the client user device may transmit the selection to the server. At step 1610, the client may receive a confirmation from the server that the caregiver has been assigned to the caregiver services request either automatically or following acceptance of the request by the caregiver.

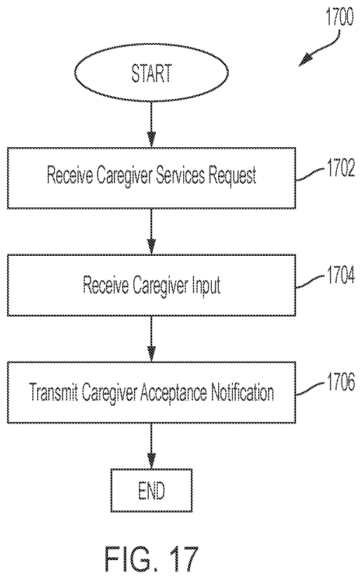

Caregivers may view and accept requests for caregiver services on caregiver user devices. An example method 1700 of accepting requests for caregiver services via a caregiver user device is shown in FIG. 17. The method 1700 may begin, at step 1702 with receipt of a caregiver services request. The caregiver services request may include information such as a nature of the request, specific tasks associated with the request, a time window in which the caregiver services are requested, specific requirements, such as certifications, associated with the request, a location of the client, and other caregiver services request information. In some embodiments, the caregiver services request may be a request that is sent to multiple caregiver user devices. When the caregiver views a map, list, or other interface, multiple caregiver services requests may be displayed and the caregiver may accept caregiver services requests from the interface. In some embodiments, caregiver services requests received by the caregiver user device and/or displayed on an interface of the caregiver user device may be limited to services for which the caregiver is verified, such as by possessing credentials associated with requirements of the request, such as specialized education, training, and/or certifications. In some embodiments the caregiver services request may be a request from a client requesting services of only a single caregiver.

At step 1704 the caregiver user device may receive caregiver input as to whether the caregiver will accept the user request. For example, for general caregiver service requests the caregiver may choose to accept or ignore requests that are displayed. In some cases where services are requested from a specific caregiver, the caregiver may be required to accept or reject the request for caregiver services. At step 1706, caregiver acceptance information, which may include notification of acceptance of a request or of rejection of a request, may then be transmitted from the caregiver user device to the server. Thus, a caregiver may operate a caregiver user device to accept and/or reject caregiver services requests.

The schematic flow chart diagrams of FIGS. 6-8 and 15-17 are generally set forth as logical flow diagrams. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method comprising:
receiving, by a server, a request for creation of a caregiver account from a first user device;
transmitting, by the server, a request for at least one credential for a caregiver associated with the caregiver account to the first user device;
receiving, by the server, the at least one credential for the caregiver from the first user device; and
creating a caregiver account for the caregiver based, at least in part, on the receipt of the at least one credential.

2. The method of claim 1, further comprising verifying, by the server, the at least one credential received from the user device.

3. The method of claim 1, wherein creating a caregiver account comprises adding caregiver information to a map interface for selection by a potential client.

4. The method of claim 1, further comprising granting the caregiver account access to a map interface showing locations of one or more clients requesting caregiver services.

5. The method of claim 1, wherein transmitting, by the server, a request comprises transmitting by the server one or more caregiver policies and a request for an electronic signature acknowledging the one or more caregiver policies.

6. The method of claim 1, further comprising:
transmitting, by the server, a push notification requesting renewal of the at least one credential to the first user device;
receiving, by the server, a renewed credential for the caregiver from the first user device; and
updating the caregiver account of the caregiver based, at least in part, on the renewed credential.

7. The method of claim 2, further comprising:
transmitting a skills training request to the user device; and
preventing the caregiver account from accessing potential work assignments until the skills training request has been fulfilled.

8. An apparatus comprising:
a memory; and
a processor;
wherein the processor is configured to perform steps comprising:
receiving, by the processor, a request for creation of a caregiver account from a first user device;
transmitting, by the processor, a request for at least one credential for a caregiver associated with the caregiver account to the first user device;
receiving, by the processor, the at least one credential for the caregiver from the first user device; and
creating a caregiver account for the caregiver based, at least in part, on the receipt of the at least one credential.

9. The apparatus of claim 8, further comprising verifying, by the processor, the at least one credential received from the user device.

10. The apparatus of claim 8, wherein creating a caregiver account comprises adding caregiver information to a map interface for selection by a potential client.

11. The apparatus of claim 8, wherein the processor is configured to perform steps further comprising granting the caregiver account access to a map interface showing locations of one or more clients requesting caregiver services.

12. The apparatus of claim 8, wherein transmitting, by the processor, a request comprises transmitting from the processor one or more caregiver policies and a request for an electronic signature acknowledging the one or more caregiver policies.

13. The apparatus of claim 8, wherein the processor is configured to perform steps further comprising:
transmitting, by the processor, a push notification requesting renewal of the at least one credential to the first user device;
receiving, by the processor, a renewed credential for the caregiver from the first user device; and
updating the caregiver account of the caregiver based, at least in part, on the renewed credential.

14. The apparatus of claim 8, wherein the processor is further configured to perform steps further comprising:
transmitting a skills training request to the user device; and
preventing the caregiver account from accessing potential work assignments until the skills training request has been fulfilled.

15. An apparatus comprising:
a memory; and
a processor;
wherein the processor is configured to perform steps comprising:
receiving, from a client user device, a request for caregiver services;
determining one or more caregiver requirements based, at least in part, on the request for caregiver services;
determining at least one caregiver to match to a client operating the client user device based, at least in part, on the one or more caregiver requirements, wherein the at least one caregiver is verified for the one or more caregiver requirements; and
transmitting, to the client user device, information regarding at the at least one caregiver for display on the client device.

16. The apparatus of claim 15, wherein the processor is configured to perform steps further comprising:
receiving, from the client user device, a selection of at least one of the one or more matched caregivers; and
transmitting, to at least one caregiver user device associated with the selected one or more caregivers, a notification that a client has requested caregiver services.

17. The apparatus of claim 16, wherein the processor is further configured to perform steps further comprising:
receiving, from the at least one caregiver user device, a notification that the caregiver has accepted the client request; and
transmitting, to the client user device, a notification that the caregiver has accepted the client request.

18. The apparatus of claim 15, wherein a caregiver that is verified for the at least one or more caregiver requirements has submitted one or more credentials corresponding to the one or more caregiver requirements.

19. The apparatus of claim 15, wherein transmitting, to the client user device, information regarding the at least one caregiver comprises transmitting location information for the at least one caregiver to be displayed on a map interface of the client user device.

20. The apparatus of claim 15, wherein the processor is further configured to perform steps further comprising:
receiving, by the processor, a request for creation of a client account from the client user device;
transmitting, by the processor, a request for at least one credential for the client associated with the client account to the client user device;
receiving, by the processor, the at least one credential for the client from the client user device; and
creating a client account for the client based, at least in part, on the receipt of the at least one credential.
